(19) United States
(12) Patent Application Publication
(10) Pub. No.: US 2015/0024406 A1
(43) Pub. Date: Jan. 22, 2015

(54) INCREASED AQUAPORIN EXPRESSION ON CELLULAR MEMBRANE TO IMPROVE CRYOPRESERVATION EFFICIENCY

(71) Applicants: Robin N. Coger-Simmons, Oak Ridge, NC (US); Laura Whritenour Schrum, Huntersville, NC (US); Balasubramanian Karthik Kumar, Charlotte, NC (US); Charles Lee, Charlotte, NC (US)

(72) Inventors: Robin N. Coger-Simmons, Oak Ridge, NC (US); Laura Whritenour Schrum, Huntersville, NC (US); Balasubramanian Karthik Kumar, Charlotte, NC (US); Charles Lee, Charlotte, NC (US)

(21) Appl. No.: 14/330,738

(22) Filed: Jul. 14, 2014

Related U.S. Application Data
(60) Provisional application No. 61/847,186, filed on Jul. 17, 2013.

(51) Int. Cl. A01N 1/02; G01N 33/50; C12N 5/071

(52) U.S. Cl. CPC A01N 1/0221; C12N 5/067; G01N 33/5067; G01N 33/5026; C12N 2501/01; C12N 2501/335; G01N 2500/10; G01N 2333/705
USPC 435/7.21; 435/374; 435/366; 435/32

(57) ABSTRACT

A method of storing mammalian cells or tissue (e.g., liver cells or hepatocytes) for subsequent use comprises the steps of: (a) contacting the cells or tissue in vitro to a choleretic agent in an effective amount; (b) combining said cells or tissue with a cryopreservative; (c) freezing said cells or tissue, and then (d) storing said frozen cells or tissue in frozen form for subsequent use.

### RELATED APPLICATIONS

This application claims the benefit of U.S. Provisional Patent Application Ser. No. 61/847,186, filed Jul. 17, 2013, the disclosure of which is incorporated by reference herein in its entirety.

### BACKGROUND OF THE INVENTION

A key step in developing a new drug for human or veterinary use is screening large numbers of drug candidates for safety, as well as effectiveness. Safety screening has traditionally been carried out with live animals (that is, in vivo). Live animal toxicity screening is, however, expensive, and objectionable to some.

Liver cells, particularly liver hepatocytes, are considered versatile tools for screening potential new drugs for toxicity in vitro. While such cells must obviously be collected from a donor, in some cases such cells can be expanded in vitro. In either case, for use in toxicity screening, the harvesting, freezing, storage/shipping, thawing and subsequent use of liver cells is often required. Accordingly, there is a need for new ways to enhance the cryopreservation efficiency of liver hepatocytes, along with other cells and tissues that may be frozen and revived, while maintaining hepatocyte function, for subsequent use in toxicity screening or the like—and even for tissue or organ transplantation.

### SUMMARY OF THE INVENTION

A first aspect of the present invention is a method of storing mammalian cells or tissue for subsequent use, comprising the steps of: (a) contacting the cells or tissue in vitro to a choleretic agent in an effective amount; (b) combining said cells or tissue with a cryopreservative; (c) freezing said cells or tissue, and then (d) storing said frozen cells or tissue in frozen form for subsequent use.

A further aspect of the present invention is frozen mammalian cells or tissues produced by a method as described above, and in further detail below, preferably in sterile form and stored in sterile form in a container.

A further aspect of the present invention is a method of providing live mammalian cells or tissue, comprising the steps of: (a) thawing cells or tissue of claim as described above, and in further detail below, for subsequent use.

The present invention is explained in greater detail in the drawings herein and the specification set forth below. The disclosures of all United States patent references cited herein are to be incorporated herein by reference in their entirety.

### BRIEF DESCRIPTION OF THE DRAWINGS

FIG. 1. Cell shrinkage analysis results. The plot shows the percentage reduction in the cross sectional area of the treated and control cells over time under the influence of the hypertonic environment. n=5, Mean±SE.

FIG. 2. Post-thaw cell viabilities for treated and control samples with DMSO as the cryoprotectant. n=4, *#%: p<0.05. Mean±SE.

FIG. 3. Post-thaw cell viabilities for treated and control samples with glycerol as the cryoprotectant agent. n=4, *#%: p<0.05. Mean±SE.

### DETAILED DESCRIPTION OF THE PREFERRED EMBODIMENTS

“Mammalian” as used herein may be any mammalian species, including, dog, cat, mouse, cow, horse, etc., as well as primate species, particularly human.

“Cells” as used herein may refer to cells separated from a tissue, or cells that reside in a tissue. In a particular embodiment the cells are liver cells, particularly hepatocytes.

“Tissue” as used herein refers to an organized assembly of different cells. For example, “liver tissue” may be comprised of hepatocytes, along with sinusoidal hepatic endothelial cells, Kupffer cells, hepatic stellate cells, etc., organized as found in vivo.

“Choleretic agent” or “choleretic stimuli” as used herein may be any compound which enhances bile secretion when administered to a mammalian subject. Numerous such compounds are known, including but not limited to those described in U.S. Pat. Nos. 3,065,134; 3,084,100; 3,309,271; 3,708,544; and 3,700,775, the disclosures of which are incorporated herein by reference. In some embodiments, glucagon or cyclic adenosine monophosphate (cAMP), including analogs thereof, are preferred choleretic agents.

“Cryopreservative” or “cryoprotectant” as used herein may be any suitable compound that protects cells from damage during freezing (e.g., due to ice crystal formation therein). Examples of known cryoprotectants include but are not limited to dimethyl sulfoxide (DMSO), polyols such as glycerol, ethylene glycol, and propylene glycol, etc. A preferred cryoprotectant is glycerol.

### 1. Cryopreserved Products and Methods of Making.

As noted above, the present invention provides a method of storing mammalian (including but not limited to human) cells or tissue for subsequent use. In particular embodiments, the cells or tissue are liver cells or tissue, and preferably comprise hepatocytes. The method generally comprises the steps of: (a) contacting the cells or tissue in vitro to a choleretic agent in an effective amount; (b) combining said cells or tissue with cryopreservative; and then (c) freezing the cells or tissue.

Suitable choleretic agents include, but are not limited to, glucagon and DiButyryl cAMP (Bt₂cAMP; e.g., bucladesine or sodium (3aR,4R,6R,6aR)-4-(6-butanamido-9H-purin-9-yl)-6-[(butanoyloxy)methyl]-2-oxo-tetrahydro-2H-1,3,5,2λ⁵-furo[3,4-d][1,3,2] dioxaphosphol-2-olate), but may be any agent that is directly or indirectly effective to increase the expression of aquaporins (AQP, particularly AQP8) on cell membranes thereof, and/or is effective to enhance or improve the water transport properties of the cells (for example, that is effective to enhance the shrinkage of said cells, or cells of said tissue, when said cells or tissue are contacted to a hypertonic solution).

Any suitable cryopreservative or cryoprotectant may be used to carry out the present invention, with glycerol currently preferred. In general, the cells or tissue are contacted to an aqueous solution containing at least one cryopreservative or cryoprotectant.

After freezing, the cells or tissue are then generally (d) stored (e.g., for a period of 1 to 2 weeks, up to 2 to 4 months or more) for subsequent use. Such use may be at the same facility, or a different facility to which the cells or tissue are shipped in frozen form (e.g., by packing with dry ice). The cells or tissues are preferably stored in sterile form in a suitable sealed container, in accordance with known techniques.

2. Methods of Use.

When needed, frozen cell or tissue products as described above may be reconstituted or revived to provide live tissue for further use. In general, the cells or tissue are (a) thawed, and (optionally but preferably), (b) rinsed to remove cryopreservative therefrom. Numerous suitable rinse solutions are known, including but not limited to those described in U.S. Pat. Nos. 5,145,771 and 6,080,730 to LeMasters and Thurman, U.S. Pat. No. 5,821,045 to Fahy et al., and U.S. Pat. No. 7,977,042 to Lee et al. Such cells or tissue may then optionally be cultured or propagated (e.g., by placing the cells in a culture or growth media in accordance with known techniques, or producing a microfluidic hepatocyte chip therewith), and used for any suitable purpose.

In one embodiment, the reconstituted cells or tissues of the invention are used in toxicology screening, typically by contacting a test compound (e.g., a drug candidate) to the cells; and then detecting death or impairment of function of the cells following said contacting step. Such testing may be carried out in accordance with known techniques, including but not limited to those described in Y.-C. Toh et al., A microfluidic 3D hepatocyte chip for drug toxicity testing. Lab Chip 9, 2026-2035 (2009).

The present invention is explained in greater detail in the following non-limiting Examples.

### EXPERIMENTAL

Intracellular ice formation (IIF) is regarded as one of the major reasons for cell death (1, 2) during cryopreservation of cells. The osmotic gradient created during the freezing process and the limitation of water permeability results in increased incidence of IIF leading to higher incidence of cell death (3, 4).

The presence of water channels, known as aquaporins, a family of integral membrane proteins, facilitates water movement due to osmotic gradients across the cell membrane (5). Of the 13 isoforms discovered so far, five of them were identified to be expressed in hepatocytes: AQP0, AQP8, AQP9, AQP11 and AQP12 (6-11). Among these, AQP8 is localized in the plasma membrane (12), intracellular vesicles and the mitochondria. Prior experimental evidence shows that AQP8 has a tendency to translocate to the cellular membrane on the influence of choleretic stimulus (13, 14). Therefore, we hypothesized that increasing the presence of aquaporin on the cellular membrane by translocation of AQP8 from the intracellular vesicles can help increase the water permeation rate—thereby reducing the amount of intracellular water during the freezing process and improve the cryopreservation success of hepatocytes.

In this study, the increase of aquaporin by treatment with DiButyly cAMP (Bt₂cAMP) or glucagon and its effect on the post-thaw viability of rat primary hepatocytes was evaluated. The translocation of AQP8 via the stimuli is evaluated by immunofluorescence staining and cell shrinkage analysis. Viability of the cells is assessed by Live-Dead staining.

### Materials and Methods

Hepatocyte isolation. Sprague-Dawley male rats weighing 150-280 g were fasted 24 hours prior to isolation and hepatocytes were isolated by collagenase perfusion method (15). In brief, the rat liver was perfused with collagenase solution for approximately 10 minutes. The hepatocytes from the digested liver were isolated by mechanical disruption and filtering through a nylon mesh (105 μm). The hepatocytes were then separated from the nonparenchymal cell fractions by centrifuge (Thermo IEC CEntra-CL3R, Thermo Scientific, MA) at 50×g for 3 minutes. The viability of the centrifuged hepatocytes was evaluated immediately using trypan blue exclusion assay (Sigma-Aldrich, St. Louis, Mo.). If the resulting viability was smaller than 90%, percoll (GE healthcare, Waukesha, Wis.) centrifugation was performed to achieve a minimum of 90% viability for the cell culture. Then the hepatocytes were re-suspended in the culture media containing DMEM (Invitrogen, Gaithersburg, Md.), sodium bicarbonate (3.7 g/L), insulin (500 U/L), epidermal growth factor (20 μg/L), hydrocortisone (7.5 mg/L), 1% (v/v) of antibiotic/antimycotic solution (JR Scientific, Woodland, Calif.) and 10% (v/v) fetal bovine serum (HyClone, Thermo Scientific, Waltham, Mass.).

Culture of hepatocytes. Collagen type I gel based single gel culture of hepatocytes in 35 mm diameter tissue culture plates were used for most of the experiments. The collagen gel was first prepared by adding 8 parts of 1.1 mg/mL PureCol collagen (Advanced BioMatrix, San Diego, Calif.) to 1 part of 10×DMEM solution. The pH was adjusted to 7.4 with 0.1N HCl and/or 0.1N NaOH. 0.5 mL of the prepared collagen was then coated on the 35 mm diameter tissue culture plates and incubated for an hour at 37° C., 5% CO₂ for gelation. Cells (2×10⁶) were seeded in each tissue culture plate, 1 ml of media was added and incubated at 37° C., 5% CO₂. The media was changed after 3 hours to remove the unattached cells and incubated for 24 hours.

Treatment of the hepatocytes. After 24 hours of incubation, hepatocytes were treated with a) DiButyryl cAMP (Bt₂cAMP) and b) glucagon (1 μM) (both Sigma-Aldrich, St. Louis, Mo.) and incubated for 12 hours. For the controls, 1 mL of normal DMEM media was added to the culture plates and incubated for the same period as the treated ones. After 12 hours of incubation, 0.1 mM HgCl₂—a water channel inhibitor for 5 min was added to a portion of the control and treated cells.

Confocal immunofluorescence. For confocal immunofluorescence experiments, collagen coated chamber slides were used instead of the tissue culture plates. Hepatocytes (5×10⁵) were plated on the collagen-coated chamber slides, and incubated at 37° C. for 4 hours. The cells were then treated with a) Bt₂cAMP, b) glucagon and c) normal DMEM media (control) and incubated for 12 hours. After the 12 hours, the cells were fixed with 2% formaldehyde for 10 minutes at room temperature and permeabilized with 0.2% Triton X-100 for 2 minutes. The cells were then treated with a blocking solution containing 3% BSA at room temperature and incubated overnight at 4° C. with goat affinity-purified AQP8 antibody (Santa Cruz Biotechnology Inc., Santa Cruz, Calif.) of dilution 1:50 in PBS. Then, the chamber slides were rinsed with PBS solution and treated with Alexa Flour 488—conjugated donkey anti-goat HRP secondary antibody (Invitrogen, Calif.) for 1 hour. The dilution of the secondary antibody used was 1:400 in PBS. Then the cells were treated with 1 μg/mL concentration of Hoechst 33342 (Molecular Probes, Eugene, Oreg.) and mounted with Pro- Long (Molecular Probes, Eugene, Oreg.). Fluorescence localization of the AQP8 was then detected by immersion oil confocal microscopy with 100× magnification lens.

Cell shrinkage analysis. Cell shrinkage analysis was performed on treated and control samples prepared on tissue culture plates as described above. One culture plate at a time was transferred to an Olympus IX70 microscope (Olympus America Inc, Pa.) mounted with a computer interfaced camera (Hamamatsu Corporation, Bridgewater, N.J.). The media from culture plate was aspirated and 1 mL of 5M NaCl solution was added. Response of the cells to the hypertonic NaCl solution was captured at 40× magnification at one minute intervals for 20 minutes. Images were processed using software MetaMorph Imaging System (Molecular Devices, Sunnyvale, Calif.). Using MetaMorp, the variations of the cross-sectional area of the cells at various sites were measured over time to analyze the shrinkage behavior of the cells in the hypertonic solution.

Cryopreservation of Treated and Control Samples. After treatment of cell cultures for 12 hours, samples were removed from the incubator and placed on ice to reduce the cell temperature to 4° C. to minimize the toxicity of the cryoprotecting agent (CPA). Two different CPA solutions, 20% dimethyl sulfoxide in DMEM media and 20% glycerol in DMEM media were used. The CPA solution (1 mL) was added to the samples and incubated at 4° C. for 10 minutes to reach equilibrium. Samples were transferred to the CryoMed Control Rate freezer (Thermo Forma, Waltham, Mass.).

The controlled cooling process was initiated at 4° C. and a cooling rate of 1° C./min was maintained until −16° C. followed by a cooling rate of 2° C./min until −36° C./min, and thereafter a cooling rate of 10° C./min until a temperature of −80° C. was achieved. The samples were further maintained at −80° C. for 5 minutes to ensure equilibrium. At the end of the freezing process, the samples were transferred immediately to a −80° C. Revco freezer (Kendro Laboratory, Ashville, N.C.) and stored for a week.

Evaluation of Post-thaw Cell Viability. The cryopreserved samples from the −80° C. freeze were transferred to a sterile glass box and plated in a water bath maintained at 37° C. until the media in the frozen samples completely thawed. At this juncture, the samples were between 5-10° C. Immediately, the CPA containing media in the samples was aspirated to prevent any toxicity to the cells. DMEM (1 mL) was added to the samples and incubated at 37° C. for 10 minutes. After 10 minutes of incubation, the media in the samples was again refreshed in order to remove any remaining traces of CPA. These samples were then placed in the incubator at 37° C., 5% CO₂ for 24 hours and allowed to recuperate from the freeze-thaw process.

After 24 hours of recuperation time, cell viability of the samples was determined by using nuclei fluorescence dyes. The samples were washed with 1× PBS solution and incubated with Hoechst Dye (1 μg/mL) and Ethidium Homodimer (2 μM)(Molecular Probes, Eugene, Oreg.) in PBS for 30 minutes. The viability solution was aspirated, and samples were fixed by adding 10% formalin (1 mL) (VWR, West Chester, Pa.) and incubating for 20 minutes. Subsequently, the cell viability was examined with a confocal microscope with DAPI (excitation 358 nm; emission 461 nm) and Texas red (excitation 596 nm; emission 620 nm) filters. The fluorescent images obtained were then analyzed using MetaMorp Imaging System.

Statistical Analysis. One-way Analysis of Variance (ANOVA) was performed to determine the significant differences for all the data analyses. All the analyses were considered a two tailed test with the type I error, α as 5%. Most of the experiments were performed for doublet samples (in some case triplicates) and each experiment was repeated for a minimum of three rats.

### Results

Confocal immunofluorescence. The images for the confocal immunofluorescence microscopy were captured for a 100× magnification. The Alex Fluor 488 labeling of AQP8 for cultured hepatocytes treated for 12 hours with a) no choleretic stimuli (control), b) 100 μM Bt₂cAMP and c) 1 μM glucagon, for cells from three different rats was observed (photographic images not shown). For controls, the distribution of AQP8 is evenly distributed throughout the cytosol and the plasma membrane, indicating AQP8 localization in the vesicles as well as the cellular membrane. In contrast, images for the cells treated with both Bt₂cAMP and glucagon show a higher density of AQP8 labeling at the cellular membrane. This suggests that these treatments led to the translocation of AQP8 from the vesicle to the cellular membrane. Also confocal immunofluorescence microscopy was performed for control samples incubated in the absence of the a) primary antibody, b) secondary antibody and c) both to check for any non-specific labeling. No non-specific fluorescent labeling was detected, confirming the integrity of the results obtained.

Cell shrinkage assay. Treated and control cells were subjected to a hypertonic environment which initiated osmotic water transport across the cellular membrane, resulting in cell shrinkage over time. The cells were monitored using a microscope—camera arrangement and images were captured at 1 minute intervals.

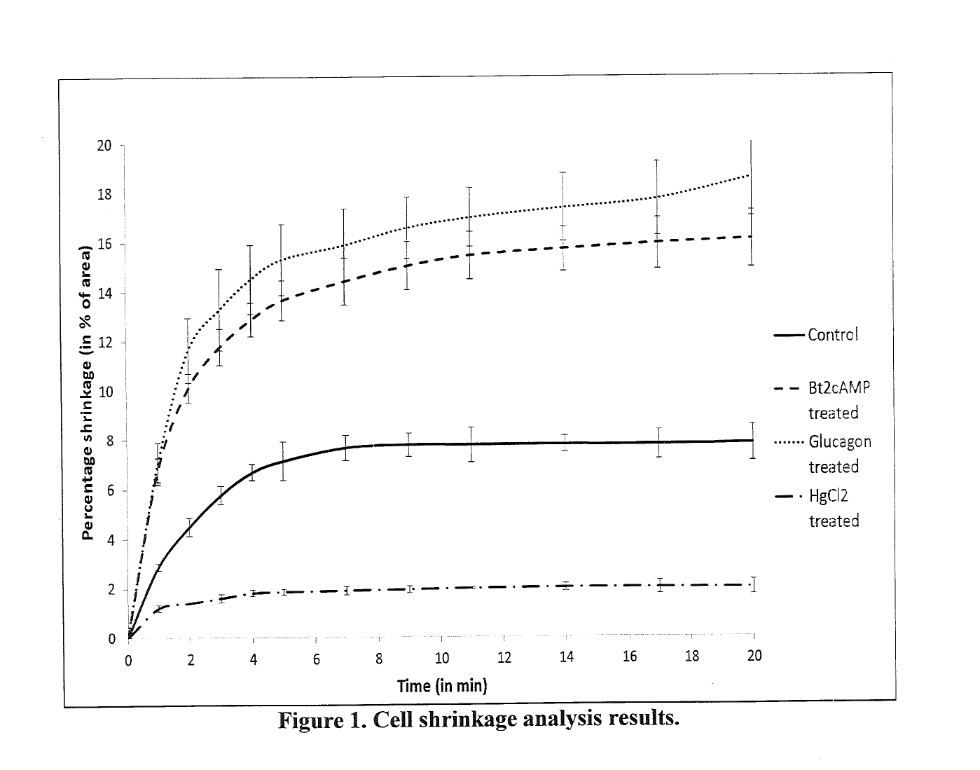

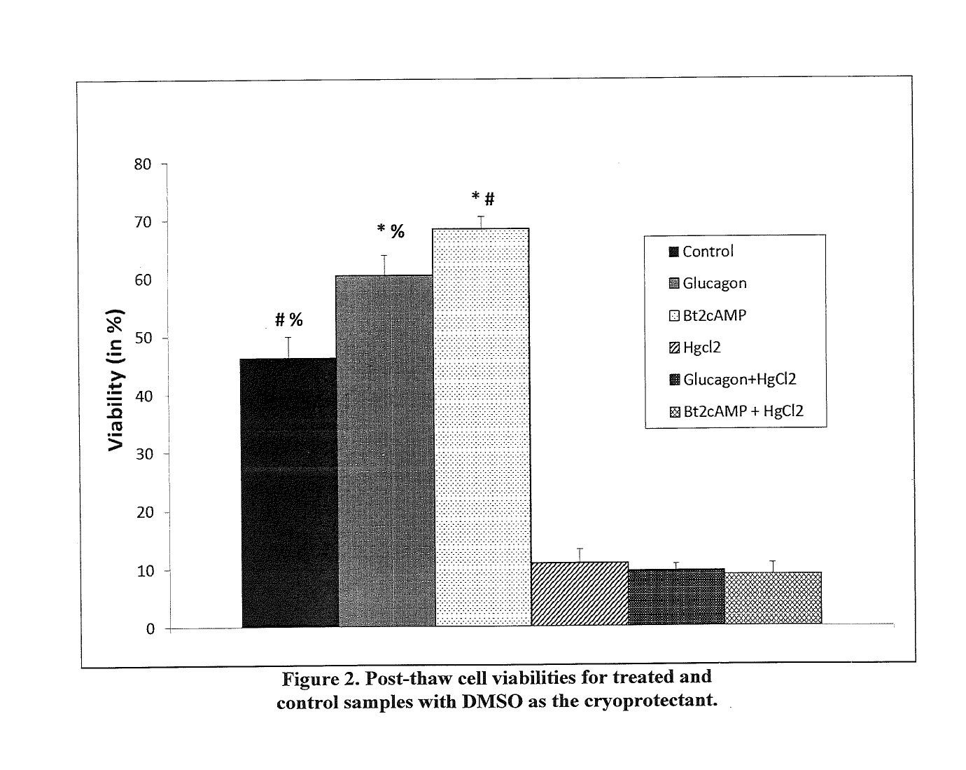

Analysis of images acquired over time for the various samples are collectively summarized in the graph shown in FIG. 1. The cells treated with Bt₂cAMP and glucagon showed a significant decrease in their cross-sectional area over time as compared to the control cells. This indicates an increase in the osmotic water transport, correlating with an increase of aquaporins at the cellular membrane. Furthermore, cells in the samples treated with HgCl₂, the water channel inhibitor, showed no significant shrinkage behavior. It holds true even for the samples first treated with Bt₂cAMP and/or glucagon and then treated with HgCl₂—not shown in FIG. 2 since their curves were mostly overlapping with the curve for the HgCl₂ treated cells. Thus, the cell shrinkage analysis suggests that the water permeability of the cells treated with Bt₂cAMP/glucagon increase mainly due to increase in the water channels (aquaporins) on the cellular membranes—the effects of which are nullified by HgCl₂.

Cell viability following Cryopreservation. All frozen samples had a storage period of one week at −80° C. The samples were incubated for 24 hours in the incubator at 37° C., 5% CO₂ prior to cell viability assessment. Images of the fluorescently stained samples were captured using confocal microscopy with DAPI (all cells) and Texas Red (dead cells) filters (images not shown).

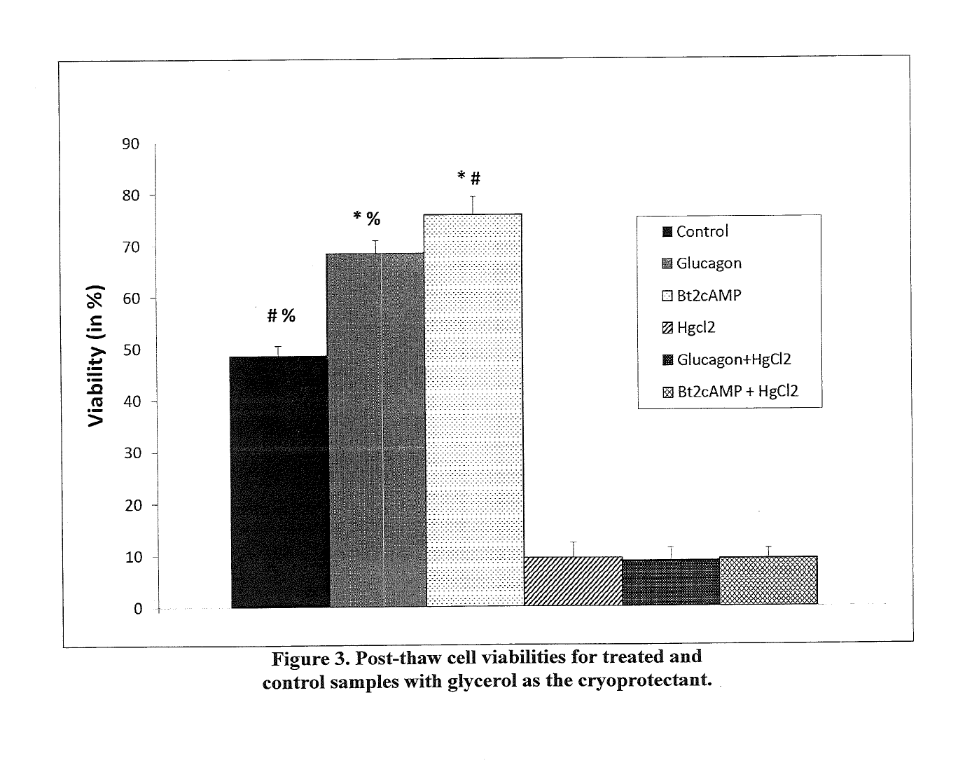

For each sample, four different fields of confocal fluorescent images were captured. Images were then analyzed using MetaMorph Imaging System which enabled a quantitative measurement of cell viability in each field. Cell viability of each sample was then estimated from the cumulative cell viabilities of the four fields per sample. The comprehensive viability assessment for the treated and control culture samples are depicted in FIG. 2 and FIG. 3.

FIG. 2 shows the post-thaw viability measurement for the samples cryopreserved with 20% DMSO in DMEM and FIG. 3 shows the samples cryopreserved with 20% glycerol in DMEM. Both cryoprotectants show similar results. The cell viability of the cultures treated with Bt₂cAMP or glucagon was significantly higher than the control. To demonstrate aquaporins increase cell viability after freeze thawing, samples treated with HgCl₂, a water channel inhibitor, had significantly lower cell viability.

Furthermore, another interesting correlation arises from the results shown in FIGS. 4 and 5. The results can be used to ascertain the effects of the CPAs used for the cryopreservation procedure in relation to the regulation of aquaporins. In the case of the cells treated with Bt₂cAMP or glucagon, a significant increase in the cell survival is observed with the use of glycerol as the CPA, whereas no such significance is seen for the other cases.

### Discussion

In the current work, the hypothesis of increasing aquaporins on hepatocyte cellular membrane by choleretic stimuli to improve cryopreservation of hepatocytes was investigated. Though similar studies have been performed for the successful cryopreservation of embryos, larvae, oocytes and kidney cells (16-20), the role of aquaporins in cryopreservation of rat primary hepatocytes has not been investigated yet. In the cases of embryos, larvae, oocyte and kidney, aquaporins were artificially expressed on the cellular membrane prior to cryopreservation. Contrastingly, in the current investigation, the fact that aquaporins can be increased in the cellular membrane by the translocation of AQP8 from the intracellular vesicles under the influence of choleretic stimuli such as DiButyly cAMP (Bt2cAMP) (14) and glucagon (12, 21, 22), was utilized. As such, it was verified by the confocal immunofluorescence which showed increased AQP8 localization at the cellular boundaries on treatment with Bt₂cAMP or glucagon.

With increase in the quantity of aquaporins on the cellular membrane, it was expected that the water transport properties of the cells also should improve. This was verified by the cell shrinkage analysis, wherein the hepatocytes cultured in a collagen gel matrix were subjected to a hypertonic environment and their shrinking behavior was monitored over time. Such an analysis differs from the traditional swell-shrink analysis in which the swell—shrink behavior of individual cells are monitored in suspension (23) or flow (24) rather than in a collagen matrix. In the case of hepatocytes embedded in an extracellular matrix (ECM), the shrinkage of the cells is considerably restricted by its attachment to the ECM and the cell-cell interactions. Despite such restrictions, a significant increase in cell shrinkage was observed by treatment of the cells with Bt₂cAMP and/or glucagon. This suggests that such treatments can potentially improve water transport in liver tissues, slices and even in whole liver.

On establishing the method of treatments and verifying the relocation of the aquaporins, cryopreservation of treated and untreated control culture samples were performed. The samples were thawed after one week, allowed to recuperate for 24 hours and then their post-thaw viability was estimated. It is to be noted that the post-thaw cell viability reported is the ratio of the estimated number of live cells to the estimated total number of cells in the culture plates after fixing the cells with 10% formalin. This does not represent the actual cell viability with respect to the 2×10⁶ cells seeded at the initiation of the culture process because some of the dead cells would have detached and washed off during the process of changing media, removing cryoprotective media, and the washing steps. In fact, it was estimated that roughly 1.4-1.7×10⁶ cells remained attached to the culture plate at the end of the fixing step. So the reported cell viability might be slightly higher than the actual cell viability. However, this factor is not critical in the current investigation since this is a comparative investigation between the treated versus control samples and also treated and controls samples were all subjected to the same experimental processes.

The results from the post-thaw viability shown in FIGS. 2 and 3 confirmed the hypothesis that the increased AQP expression on the cellular membrane significantly improves the cryopreservation success. In addition, most of the culture samples treated with Bt₂cAMP exhibited higher cell viability compared to the ones treated with glucagon. This may be explained by treatment time or exposure time to glucagon. Treatment with glucagon for 12 hrs may not be sufficient for maximum relocation of the AQP8s. Literature suggests that the longer the cells are treated with glucagon, the more AQP8 translocates to the cellular membrane. Results from Soria et al [12] suggested that treatment of hepatocytes with glucagon for 36 hours showed 120% increase in the quantity of AQP8 on cellular membrane as opposed to 80% increase for a 16 hours treatment. On contrary, for Bt₂cAMP, some researchers indicate 10 min incubation is enough for effective translocation of AQP8 (6) whereas others recommend 12 hours (14). Therefore, there is a need for better understanding of the mechanism of AQP8 translocation and optimization of the time scale of the treatments with Bt₂cAMP and glucagon.

Finally, the CPA selected may affect the cryopreservation outcome. In the current investigation, use of glycerol as the CPA showed significantly higher post-thaw cell viability compared to DMSO. A few probable explanations can be provided for preference of glycerol over DMSO. Firstly, DMSO has been identified as a water channel blocker (14, 25). So, use of DMSO as CPA might in fact retard the water transport through the aquaporin water channels to some extent, thus exhibiting lower post-thaw viability. Secondly, AQP9 is an aquaglyceropin, which facilitates the transport of glycerol across the cellular membrane (13, 26). As a result, it might aid in better protection of hepatocytes from freeze injuries.

Overall, the current investigation was able to successfully confirm the hypothesis that translocation of aquaporins can indeed improve cryopreservation of hepatocytes. Furthermore, glycerol was identified as a preferred CPA for safe storage of hepatocytes with enhanced aquaporin localization to the cellular membrane.

### REFERENCES

1. Harris C L, Toner M, Hubel A, Cravalho E G, Yarmush M L, Tompkins R G. Cryopreservation of isolated hepatocytes: intracellular ice formation under various chemical and physical conditions. Cryobiology 1991; 28 (5): 436.

2. Hubel A, Toner M, Cravalho E G, Yarmush M L, Tompkins R G. Intracellular ice formation during the freezing of hepatocytes cultured in a double collagen gel. Biotechnol Prog 1991; 7 (6): 554.

3. Karlsson J O, Cravalho E G, Borel Rinkes I H, Tompkins R G, Yarmush M L, Toner M. Nucleation and growth of ice crystals inside cultured hepatocytes during freezing in the presence of dimethyl sulfoxide. Biophys J 1993; 65 (6): 2524.

4. Wolkers W F, Balasubramanian S K, Ongstad E L, Zec H C, Bischof J C. Effects of freezing on membranes and proteins in LNCaP prostate tumor cells. Biochim Biophys Acta 2007; 1768 (3): 728.

5. Verkman A S, van Hoek A N, Ma T, et al. Water transport across mammalian cell membranes. Am J Physiol 1996; 270 (1 Pt 1): C12.

6. Huebert R C, Splinter P L, Garcia F, Marinelli R A, LaRusso N F. Expression and localization of aquaporin water channels in rat hepatocytes. Evidence for a role in canalicular bile secretion. J Biol Chem 2002; 277 (25): 22710.

7. Calamita G, Mazzone A, Bizzoca A, et al. Expression and immunolocalization of the aquaporin-8 water channel in rat gastrointestinal tract. Eur J Cell Biol 2001; 80 (11): 711.

8. Elkjaer M L, Nejsum L N, Gresz V, et al. Immunolocalization of aquaporin-8 in rat kidney, gastrointestinal tract, testis, and airways. Am J Physiol Renal Physiol 2001; 281 (6): F1047.

9. Tani T, Koyama Y, Nihei K, et al. Immunolocalization of aquaporin-8 in rat digestive organs and testis. Arch Histol Cytol 2001; 64 (2): 159.

10. Elkjaer M, Vajda Z, Nejsum L N, et al. Immunolocalization of AQP9 in liver, epididymis, testis, spleen, and brain. Biochem Biophys Res Commun 2000; 276 (3): 1118.

11. Nicchia G P, Frigeri A, Nico B, Ribatti D, Svelto M. Tissue distribution and membrane localization of aquaporin-9 water channel: evidence for sex-linked differences in liver. J Histochem Cytochem 2001; 49 (12): 1547.

12. Soria L R, Gradilone S A, Larocca M C, Marinelli R A. Glucagon induces the gene expression of aquaporin-8 but not that of aquaporin-9 water channels in the rat hepatocyte. Am J Physiol Regul Integr Comp Physiol 2009; 296 (4): R1274.

13. Gradilone S A, Carreras F I, Lehmann G L, Marinelli R A. Phosphoinositide 3-kinase is involved in the glucagon-induced translocation of aquaporin-8 to hepatocyte plasma membrane. Biol Cell 2005; 97 (11): 831.

14. Garcia F, Kierbel A, Larocca M C, et al. The water channel aquaporin-8 is mainly intracellular in rat hepatocytes, and its plasma membrane insertion is stimulated by cyclic AMP. J Biol Chem 2001; 276 (15): 12147.

15. Seglen P. Preparation of isolated rat liver cells. Methods Cell Biol 1976; 13: 29.

16. Wang W, Ben R N. Upregulation and protein trafficking of aquaporin-2 attenuate cold-induced osmotic damage during cryopreservation. In Vitro Cell Dev Biol Anim 2004; 40 (3-4): 67.

17. Edashige K, Yamaji Y, Kleinhans F W, Kasai M. Artificial expression of aquaporin-3 improves the survival of mouse oocytes after cryopreservation. Biol Reprod 2003; 68 (1): 87.

18. Hagedorn M, Lance S L, Fonseca D M, et al. Altering fish embryos with aquaporin-3: an essential step toward successful cryopreservation. Biol Reprod 2002; 67 (3): 961.

19. Van den Abbeel E, Schneider U, Liu J, Agca Y, Critser J K, Van Steirteghem A. Osmotic responses and tolerance limits to changes in external osmolalities, and oolemma permeability characteristics, of human in vitro matured MII oocytes. Hum Reprod 2007; 22 (7): 1959.

20. Philip B N, Yi S X, Elnitsky M A, Lee R E, JR. Aquaporins play a role in desiccation and freeze tolerance in larvae of the goldenrod gall fly, Eurosta solidaginis. J Exp Biol 2008; 211 (Pt 7): 1114.

21. Gradilone S A, Tietz P S, Splinter P L, Marinelli R A, LaRusso N F. Expression and subcellular localization of aquaporin water channels in the polarized hepatocyte cell line, WIF-B, BMC Physiol 2005; 5: 13.

22. Gradilone S A, Garcia F, Huebert R C, et al. Glucagon induces the plasma membrane insertion of functional aquaporin-8 water channels in isolated rat hepatocytes. Hepatology 2003; 37 (6): 1435.

23. Majno G, Joris I. Apoptosis, oncosis, and necrosis. An overview of cell death. Am J Pathol 1995; 146 (1): 3.

24. Stoll B, Gerok W, Lang F, Haussinger D. Liver cell volume and protein synthesis. Biochem J 1992; 287 (Pt 1): 217.

25. Gradilone S A, Ochoa J E, Garcia F, Larocca M C, Pellegrino J M, Marinelli R A. Hepatocyte membrane water permeability measured by silicone layer filtering centrifugation. Anal Biochem 2002; 302 (1): 104.

26. Carbrey J M, Gorelick-Feldman D A, Kozono D, Praetorius J, Nielsen S, Agre P. Aquaglyceroporin AQP9: solute permeation and metabolic control of expression in liver. Proc Natl Acad Sci USA 2003; 100 (5): 2945.

The foregoing is illustrative of the present invention, and is not to be construed as limiting thereof. The invention is defined by the following claims, with equivalents of the claims to be included therein.

That which is claimed is:

1. A method of storing mammalian cells or tissue for subsequent use, comprising the steps of:
(a) contacting the cells or tissue in vitro to a choleretic agent in an effective amount;
(b) combining said cells or tissue with a cryopreservative; and then
(c) freezing said cells or tissue.

2. The method of claim 1, further comprising the step of:
(d) storing said frozen cells or tissue in frozen form for subsequent use.

3. The method of claim 1, wherein said cells or tissue are liver cells or tissue.

4. The method of claim 1, wherein said cells or tissue comprise hepatocytes.

5. The method of claim 1, wherein said choleretic agent comprises DiButyryl cAMP (Bt₂cAMP) or glucagon.

6. The method of claim 1, wherein said cryopreservative comprises glycerol.

7. The method of claim 1, wherein said mammalian cells or tissue are human cells or tissue.

8. The method of claim 1, wherein said choleretic agent is contacted to said cells or tissue in an amount effective to increase the expression of aquaporins on cell membranes thereof.

9. The method of claim 8, wherein said aquaporins comprise Aquaporin-8 (AQP8).

10. The method of claim 2, wherein said storing step is carried out for at least one week.

11. Frozen mammalian cells or tissues produced by the process of claim 1 and stored in sterile form in a container.

12. A method of providing live mammalian cells or tissue, comprising the step of:
(a) thawing cells or tissue of claim 11.

13. The method of claim 12, further comprising the step of:
(b) rinsing said cells or tissue with an aqueous solution to remove cryopreservative therefrom.

14. The method of claim 13, further comprising the step of:
(c) culturing or propagating said cells.

15. The method of claim 14, further comprising the steps of:
(d) contacting a test compound to said cells; and then
(e) detecting death or impairment of function of said cells following said contacting step.
